(19) United States
(12) Patent Application Publication
(10) Pub. No.: US 2018/0042537 A1
(43) Pub. Date: Feb. 15, 2018

(54) Wireless Wearable Brain Blood-Oxygen Monitoring System

(71) Applicant: Institute of Automation Chinese Academy of Sciences, BEIJING (CN)

(72) Inventors: Xin Zhang, Beijing (CN); Nianming Zuo, Beijing (CN); Tianzai Jiang, Beijing (CN); Ruirui Zhao, Beijing (CN)

(21) Appl. No.: 15/555,525

(22) PCT Filed: Mar. 3, 2015
(86) PCT No.: PCT/CN2015/073585

§ 371 Date: Sep. 4, 2017

(51) Int. Cl. A61B 5/1455; A61B 5/00

(52) U.S. Cl. CPC A61B 5/14553; A61B 5/0013; A61B 5/0022; A61B 5/6803; A61B 5/7225; A61B 5/742; A61B 5/7475; A61B 2562/0238; A61B 2560/0214; A61B 2562/164; A61B 2562/166

(57) ABSTRACT

The wireless wearable brain blood-oxygen monitoring system provided by the present Application comprises a monitoring control device, a detection device, a fixing device and a flexible printed circuit board; said monitoring control device is used for processing a first optical signal to generate a digital signal and sending said digital signal to a portable mobile terminal; said detection device is used for receiving the first optical signal sent by said monitoring control device, and calculating a degree of blood-oxygen saturation in a detected area according to an incident light intensity and an emergent light intensity of the first optical signal; said flexible printed circuit board is used for fixing a photoelectric conversion means in said detection device; said fixing device is used for fitting said monitoring control device and said detection device to the forehead. The present Application can make the brain blood-oxygen monitoring system to monitor in real time and can make it portable.

### TECHNICAL FIELD

The present Application relates to the technical field of near infrared spectroscopy brain imaging, in particular to a wireless wearable brain blood-oxygen monitoring system.

### BACKGROUND OF THE APPLICATION

Brain imaging technology has become a new focus of research in cognitive neuroscience, and a lot of new technologies and new Applications emerge, among which a new brain blood-oxygen monitoring technology is the near infrared spectroscopy brain imaging technology.

Every person is exposed to the threat of diseases in modern society, and as people are paying more and more attention to their health, wearable medical devices are more widely needed, and health medical devices become essential consumer goods. Currently, the brain blood-oxygen monitoring device is too large to enable wearable measurement. Therefore, with the development of wearable medical devices, there is an urgent need for a wearable brain blood-oxygen monitoring system.

### SUMMARY OF THE APPLICATION

A wireless wearable brain blood-oxygen monitoring system provided by the present Application can enable a brain blood-oxygen monitoring system to monitor in real time and can make it portable.

According to one aspect of the present Application, a wireless wearable brain blood-oxygen monitoring system is provided, which comprises a monitoring control device, a detection device, a fixing device and a flexible printed circuit board; said monitoring control device is used for processing a first optical signal to generate a digital signal and sending said digital signal to a portable mobile terminal; said detection device is used for receiving the first optical signal sent by said monitoring control device, and calculating a degree of blood-oxygen saturation in a detected area according to an incident light intensity and an emergent light intensity of the first optical signal;

said flexible printed circuit board is used for fixing a photoelectric conversion means in said detection device;

said fixing device is used for fitting said monitoring control device and said detection device to the forehead.

The wireless wearable brain blood-oxygen monitoring system provided by the present Application can fit the monitoring control device and the detection device to the forehead through the fixing device and can send digital signals to the portable mobile terminal through the monitoring control device, so that the brain blood-oxygen monitoring system can monitor in real time and become portable.

### BRIEF DESCRIPTION OF THE DRAWINGS

FIG. 1 is a schematic drawing of a wireless wearable brain blood-oxygen monitoring system provided by an embodiment of the present Application;

FIG. 2 is a schematic drawing of a monitoring control device provided by an embodiment of the present Application;

FIG. 3 is a schematic drawing of a wireless wearable brain blood-oxygen control system provided by an embodiment of the present Application;

FIG. 4 is a schematic drawing of an application scenario of a wireless wearable brain blood-oxygen monitoring system provided by an embodiment of the present Application.

### DETAILED DESCRIPTION OF THE APPLICATION

A general idea of the present Application is to fit the monitoring control device and the detection device to the forehead using the fixing device, and to send digital signals to the portable mobile terminal using the monitoring control device, so that the brain blood-oxygen monitoring system can monitor in real time and become portable.

The wireless wearable brain blood-oxygen monitoring system provided by an embodiment of the present Application will be described in detail below in conjunction with the drawings.

FIG. 1 is a schematic drawing of a wireless wearable brain blood-oxygen monitoring system provided by an embodiment of the present Application.

Referring to FIG. 1, the system comprises a monitoring control device 30, a detection device 20, a fixing device 10, a flexible printed circuit board 40 and a light source 50.

The monitoring control device 30 is used for processing a first optical signal to generate a digital signal and sending said digital signal to a portable mobile terminal.

The detection device 20 is used for receiving the first optical signal sent by the monitoring control device 30, and calculating a degree of blood-oxygen saturation in a detected area according to an incident light intensity and an emergent light intensity of the first optical signal.

The flexible printed circuit board 40 is used for fixing a photoelectric conversion means in the detection device 20.

The wireless wearable blood-oxygen monitoring system herein comprises two detection devices 20, which respectively include a photoelectric conversion means that is at an outer side of the forehead of the head of a human body.

The fixing device 10 is used for fitting the monitoring control device 30 and the detection device 20 to the forehead.

The fixing device 10 herein is a headband.

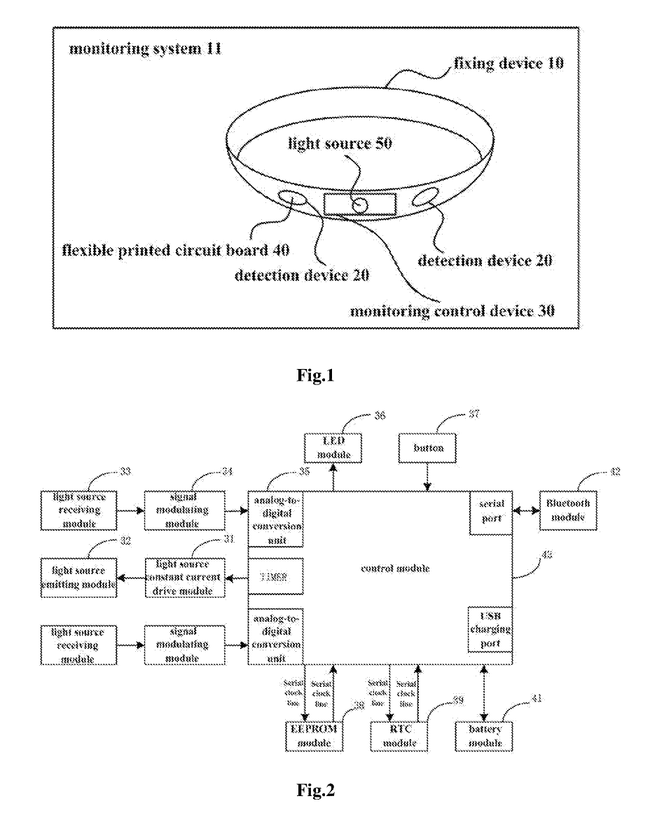

FIG. 2 is a schematic drawing of a monitoring control device provided by an embodiment of the present Application.

Referring to FIG. 2, said monitoring control device 30 comprises: a light source receiving module 33, a signal modulating module 34, a control module 43 that includes an analog-to-digital conversion unit 35, a light source emitting module 32, and a light source constant current drive module 31;

The control module 43 is used for sending control command information to the light source constant current drive module 31 to determine an emission frequency of the first optical signal.

The light source constant current drive module 31 is used for receiving the control command information sent by the control module 43 and sending the first optical signal to the light source emitting module 32 at the emission frequency.

The first optical signal herein is near infrared that is scattered and absorbed by the brain and carries the blood-oxygen information of the brain.

Emission of the light source can be controlled by a single chip machine whose power and frequency is flexibly adjustable, so emission of the first optical signal becomes more stable and controllable; besides, the light source emission module emits near infrared at a constant power and frequency, so reliability of data is guaranteed.

The light source emitting module 32 is used for receiving the first optical signal sent by the light source constant current drive module 31 and sending the first optical signal to the light source receiving module 33.

The light source receiving module 33 is used for receiving the first optical signal sent by the light source emitting module 32 and sending said first optical signal to the signal modulating module 34.

The signal modulating module 34 is used for receiving the first optical signal sent by the light source receiving module 33, converting the first optical signal into a first electrical signal, and filtering the first electrical signal to generate a second electrical signal.

The analog-to-digital conversion unit 35 is used for converting the second electrical signal into a digital signal.

According to an exemplary embodiment of the present Application, the monitoring control device 30 further comprises a Bluetooth module 42, an EEPROM module 38, a battery module 41 and an LED module 36.

The Bluetooth module 42 is used for receiving the digital signal sent by the control module 43 and sending the digital signal to a portable mobile terminal 60.

The EEPROM module 38 is used for storing data and for erasing or programming the stored data.

The battery module 41 is used for supplying a voltage and monitoring the power supply state.

The LED module 36 is used for displaying the button state.

A RTC module 39 is used for recording data in real time.

A button 37 is used for controlling ON and OFF of the system.

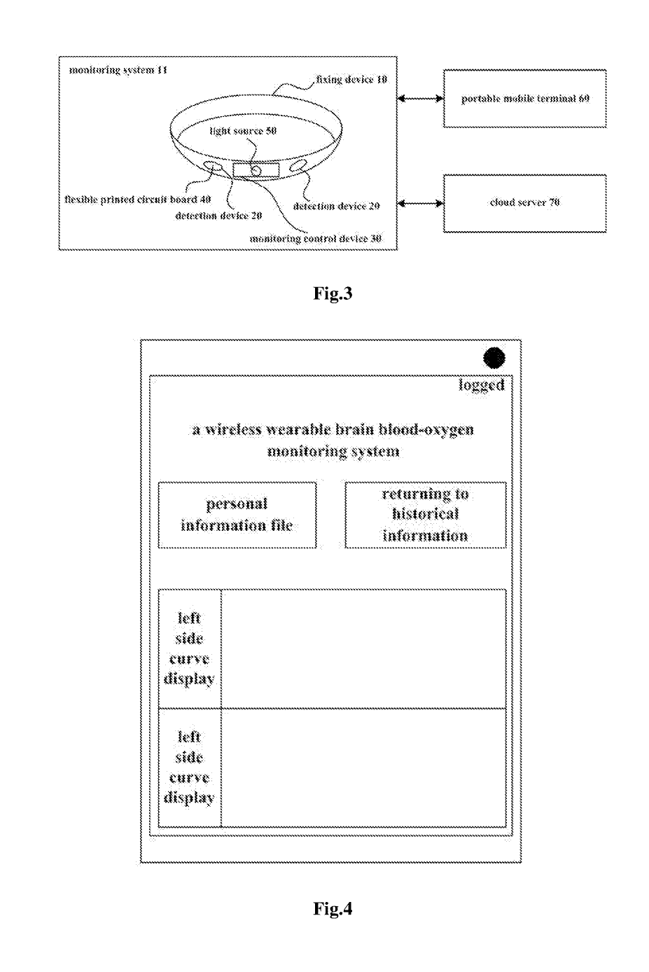

FIG. 3 is a schematic drawing of a wireless wearable brain blood-oxygen control system provided by an embodiment of the present Application.

Referring to FIG. 3, the control system comprises a monitoring system 11, a portable mobile terminal 60 and a cloud server 70. The monitoring system 11 comprises the monitoring control device 30, the detection device 20, the fixing device 10, the flexible printed circuit board 40 and the light source 50.

The portable mobile terminal 60 is used for receiving the digital signal sent by the monitoring control device 30, processing the digital signal and displaying the processed digital signal, wherein the processed digital signal is the brain blood-oxygen information of a human body; or the portable mobile terminal 60 receives data sent from the cloud server 70 and synchronizes the received data.

FIG. 4 is a schematic drawing of an application scenario of a wireless wearable brain blood-oxygen monitoring system provided by an embodiment of the present Application.

Referring to FIG. 4, firstly a system interface can be entered by logging into the wireless wearable brain blood-oxygen monitoring system; then by clicking on a personal information file, the current brain blood-oxygen information can be obtained, or by clicking on “returning to historical information”, the historical brain blood-oxygen information can be obtained.

The above described are merely preferred embodiments of the present Application, but the protection scope of the present Application is not limited to this. Any variation or replacement that is readily conceivable by those skilled in the art within the technical scope disclosed by the present Application shall fall into the protection scope of the present Application. Therefore, the protection scope of the present Application shall be determined by the protection scope of the claims.

What is claimed is:

1. A wireless wearable brain blood-oxygen monitoring system, characterized in that wireless detection of brain blood-oxygen is realized by means of wireless communication and near infrared spectroscopy technology, said system comprises a monitoring control device, a detection device, a fixing device and a flexible printed circuit board;
said monitoring control device is used for processing a first optical signal to generate a digital signal and sending said digital signal to a portable mobile terminal;
said detection device is used for receiving the first optical signal sent by said monitoring control device, and calculating a degree of blood-oxygen saturation in a detected area according to an incident light intensity and an emergent light intensity of the first optical signal;
said flexible printed circuit board is used for fixing a photoelectric conversion means in said detection device;
said fixing device is used for fitting said monitoring control device and said detection device to the forehead.

2. The system according to claim 1, characterized in that said monitoring control device comprises: a light source receiving module, a signal modulating module, a control module that includes an analog-to-digital conversion unit, a light source emitting module, and a light source constant current drive module;
the control module is used for sending control command information to the light source constant current drive module 31 to determine an emission frequency of the first optical signal;
the light source constant current drive module is used for receiving the control command information sent by the control module and sending the first optical signal to the light source emitting module at the emission frequency;
the light source emitting module is used for receiving the first optical signal sent by the light source constant current drive module and sending the first optical signal to the light source receiving module;
the light source receiving module is used for receiving the first optical signal sent by the light source emitting module and sending said first optical signal to the signal modulating module;
the signal modulating module is used for receiving the first optical signal sent by the light source receiving module, converting the first optical signal into a first electrical signal, and filtering the first electrical signal to generate a second electrical signal;
the analog-to-digital conversion unit is used for converting the second electrical signal into a digital signal.

3. The system according to claim 2, characterized in that said monitoring control device further comprises a Bluetooth module, an EEPROM module, a battery module and an LED module;
the Bluetooth module is used for receiving the digital signal sent by the control module and sending the digital signal to a portable mobile terminal;
the EEPROM module is used for storing data and for erasing or programming the stored data;
the battery module is used for supplying a voltage and monitoring the power supply state;
the LED module is used for displaying the button state.
